(19) United States
(12) Patent Application Publication
(10) Pub. No.: US 2023/0212124 A1
(43) Pub. Date: Jul. 6, 2023

(54) SMALL-MOLECULE INHIBITORS OF THE FRS2-FGFR INTERACTION

(71) Applicants: UNIVERSITÄT ZÜRICH, Zürich (CH); ETH ZÜRICH, Zürich (CH)

(72) Inventors: Gisbert SCHNEIDER, Zürich (CH); Cyrill BRUNNER, Greifensee (CH); Martin BAUMGARTNER, Rubigen (CH); Karthiga Santhana KUMAR, Zürich (CH); Oliver ZERBE, Zürichch (CH)

(73) Assignees: UNIVERSITÄT ZÜRICH, Zürich (CH); ETH ZÜRICH, Zürich (CH)

(21) Appl. No.: 18/001,075

(22) PCT Filed: Jun. 8, 2021
(86) PCT No.: PCT/EP2021/065340

§ 371 Date: Dec. 8, 2022

(30) Foreign Application Priority Data
Jun. 8, 2020 (EP) 20178685.2

(51) Int. Cl. C07D 215/233; A61P 35/04

(52) U.S. Cl. CPC C07D 215/233; A61P 35/04

(57) ABSTRACT

The present invention relates to small-molecule inhibitors of the FRS2-FGFR interaction. The present invention relates the small-molecule inhibitors for use as a medicament and for use in cancer treatment or prevention.

The present invention relates to small-molecule inhibitors of the FRS2-FGFR interaction. The present invention relates the small-molecule inhibitors for use as a medicament and for use in cancer treatment or prevention.

### BACKGROUND OF THE INVENTION

Metastasis, the dissemination and growth of neoplastic cells in an organ distant from that in which they originated, causes as much as 90% of cancer-associated mortality. Effective cancer therapy is largely dependent on the capability to prevent metastasis specifically and less toxic, targeted anti-metastatic therapies are urgently needed. An important and fundamental cause of metastasis in the majority of all solid tumours is the deregulated motile behaviour of the cancer cells. The microenvironment shapes cell behaviour and determines metastatic outcomes of tumours. Kumar et al. (Cell Reports, 2018, vol. 23, issue 13, P 3798-3812) addressed how microenvironmental cues control tumour cell invasion in paediatric brain tumour, medulloblastoma (MB). They show that bFGF promotes MB tumour cell invasion through FGF receptor (FGFR) in vitro and that blockade of FGFR represses brain tissue infiltration in vivo. TGF-β regulates pro-migratory bFGF function in a context-dependent manner. Under low bFGF, the non-canonical TGF-β pathway causes ROCK activation and cortical translocation of ERK1/2, which antagonizes FGFR signalling by inactivating FGFR substrate 2 (FRS2), and promotes a contractile, non-motile phenotype. Under high bFGF, negative-feedback regulation of FRS2 by bFGF-induced ERK1/2 causes repression of the FGFR pathway. Under these conditions, TGF-β counters inactivation of FRS2 and restores pro-migratory signalling. These findings pinpoint coincidence detection of bFGF and TGF-β signalling by FRS2 as a mechanism that controls tumour cell invasion. Thus, targeting FRS2 represents an emerging strategy to abrogate aberrant FGFR signalling.

Based on the above-mentioned state of the art, the objective of the present invention is to provide means and methods to provide small-molecule inhibitors of the FRS2-FGFR interaction. This objective is attained by the subject-matter of the independent claims of the present specification.

### SUMMARY OF THE INVENTION

A first aspect of the invention relates to a compound of the general formula (500) for use in treatment or prevention of metastasis

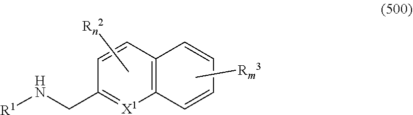

wherein
   X¹ is selected from N, O, and S, particularly X¹ is N,
   R¹ is selected from a (linear or branched) C₁-C₁₆ alkyl, (linear or branched) C₂-C₁₆ alkene, heteroaryl, aryl, a C₄-C₇ cyclo-alkyl, and a C₃-C₆ heterocycle, wherein R¹ is unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and unsubstituted or substituted C₁-C₅ alkyl or C₂-C₅ alkene, particularly R¹ is substituted with one moiety selected from OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
   each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
   n is 0, 1, 2, or 3, particularly n is 1;
   m is 0, 1, 2, 3, or 4, particularly m is 1 or 2.

A second aspect of the invention relates to a compound as described in the first aspect for use as an angiogenesis antagonist. In certain embodiments, the angiogenesis antagonist is provided in treatment or prevention of cancer. In certain embodiments, the cancer is selected from bladder cancer, hepatocellular carcinoma, and prostate cancer.

A third aspect of the invention relates to a compound as described in the first aspect for use in prevention or treatment of an FGFR-driven disease, where a transient or chronic pathological condition is induced by FGFR signaling. FGFRS are receptor tyrosine kinases involved in cell proliferation, cell differentiation, cell migration, and cell survival. Genetic alterations like gene amplifications, activating mutations and chromosomal translocations in FGFR signaling pathway have been implicated in a variety of tumour types, developmental and skeletal diseases.

A fourth aspect of the invention relates to a compound of the general formula (700)

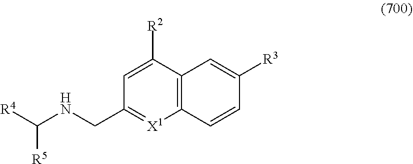

wherein
   each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
   R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
      or
   R⁴ and R⁵ together form an unsubstituted or OH—, halogen-, and/or CN-substituted cyclo-pentane or cyclo-hexane;
   X¹ is selected from N, O, and S, particularly X¹ is N,
   with the proviso that the compound is not characterized by the formula (001),

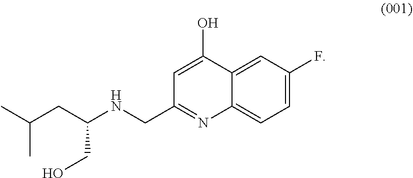 [SMILES: CC(C)C[C@@H](CO)NCc1cc(O)c2cc(F)ccc2n1]

A fifth aspect of the invention relates to a compound according to the fourth aspect for use as a medicament with the proviso that the compound includes the compound characterized by formula (001),

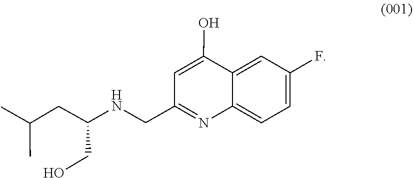 [SMILES: CC(C)C[C@@H](CO)NCc1cc(O)c2cc(F)ccc2n1]

A sixth aspect of the invention relates to a compound according to the fourth aspect for use in treatment or prevention of cancer with the proviso that the compound includes the compound characterized by the formula (001).

In another embodiment, the present invention relates a pharmaceutical composition comprising at least one of the compounds of the present invention or a pharmaceutically acceptable salt thereof and at least one pharmaceutically acceptable carrier, diluent or excipient.

The transmission of signals from activated fibroblast growth factor receptor (FGFR) tyrosine kinases promotes oncogenic functions in tumor cells, including proliferation, survival and cell migration and invasion. The interruption of signal transmission from activated FGFRS to downstream signal transduction cascades by kinase inhibitors designed against FGFRS is an established means of attenuating these oncogenic functions. In addition to aberrant activation of FGFRS in numerous malignancies, FGFR activation is also observed to act as an evasion mechanism in cancers of patients subjected to targeted therapies with kinase inhibitors, which results in tumor re-growth and progression. The small molecule compounds described in this application will prevent signal transmission from activated FGFRS to downstream effector molecules, specifically to the mitogen activated protein kinases (MAPKs), a key driver of tumorigenesis.

The compounds bind to FRS2. FGFR substrate 2 (FRS2) is a key adaptor protein that is largely specific to FGF signalling pathway. It is an exclusive downstream effector of FGFRs. FRS2 interacts with the FGFRS via the c-terminal phospho-tyrosine binding (PTB) domain and serves as a molecular hub by assembling both positive and negative signalling proteins to mediate important FGF-induced cellular functions. It transmits the signal from the FGFRs (outside of the cell) to the inside of the cell. Hence, targeting FRS2, which is very upstream of the FGF signalling pathway, effectively shuts down the downstream effectors, especially MAPKs of FGFR signaling.

The compounds specifically bind to the phosphotyrosine binding (PTB) domain of the FRS2 protein (FIG. 7). Compound binding induces a conformational shift in the PTB domain that prevents FGFR-induced signal transmission through FRS2. Two potential binding sites were initially selected: Binding site 1 is not involved in FGFR binding and located below the interaction site of FGFR's N-terminus with FRS2. Binding site 2 is the extended surface area interacting with FGFR's C-terminal end.

The mechanism of compound-target interaction, conformational change in the target domain and transmission blockade is unique and does not depend on receptor tyrosine kinase inhibition. In addition, unlike FGFRS, FRS2 does not have any shared protein domains with other adapter proteins. Thus, compared to the existing kinase inhibitors, much less off-target activity is expected. In contrast to existing FGFR targeting strategies, the compounds also interfere specifically with those FGFR functions that are particularly relevant for tumorigenesis and tumor progression.

In contrast to existing FGFR targeting strategies, the compounds also interfere specifically with those FGFR functions that are particularly relevant for tumorigenesis and tumor progression, such as proliferation, migration and invasion and angiogenesis. There is evidence of the FRS2-FGFR interaction being altered in many types of cancer, for example in prostate cancer (Yang, F. et al. Cancer Res 73, 3716-3724, 2013, Liu J et al. Oncogene. 2016 Apr. 7; 35(14):1750-9), esophageal cancer (Nemoto, T., Ohashi, K., Akashi, T., Johnson, J. D. & Hirokawa, K. Pathobiology 65, 195-203, 1997), thyroid cancer (St Bernard, R. et al. Endocrinology 146, 1145-1153, 2005), hepatocellular carcinoma (Zheng, N., Wei, W. Y. & Wang, Z. W. Transl Cancer Res 5, 1-6, 2016, Matsuki M et al. Cancer Med. 2018 June; 7(6):2641-2653), testicular cancer (Jiang, X. et al. J Diabetes Res, 2013), medulloblastoma (Santhana Kumar, K. et al. Cell Rep 23, 3798-3812 e3798, 2018), rhabdomyosarcoma (Goldstein, M., Meller, I. & Orr-Urtreger, A. Gene Chromosome Canc 46, 1028-1038, 2007), gastric cancer (Kunii, K. et al. Cancer Res 68, 3549-3549, 2008), pulmonary pleomorphic carcinoma (Lee, S. et al. J Cancer Res Clin 137, 1203-1211, 2011), breast cancer (Penaultllorca, F. et al. Int J Cancer 61, 170-176, 1995), non-small cell lung cancer (Dutt, A. et al. Plos One 6, 2011), Liposarcoma (Zhang, K. Q. et al. Cancer Res 73, 1298-1307, 2013), cervical cancer (Jang, J. H., Shin, K. H. & Park, J. G. Cancer Res 61, 3541-3543, 2001), colorectal cancer (Sato, T. et al. Oncol Rep 21, 211-216, 2009), melanoma (Becker, D., Lee, P. L., Rodeck, U. & Herlyn, M. Oncogene 7, 2303-2313, 1992), multiple myeloma (Kalff, A. & Spencer, A. Blood Cancer J, 2, 2012), endometrial cancer (Konecny, G. E. et al. Mol Cancer Ther 12, 632-642, 2013), bladder cancer (Cappellen, D. et al. Nat Genet 23, 18-20, 1999, Wu S et al. Nat Commun. 2019 Feb. 12; 10(1):720), glioblastoma (Morrison, R. S. et al. Cancer Res 54, 2794-2799, 1994), squamous cell carcinoma of the lung (Weiss, J. et al. Sci Transl Med 4, 2012), ovarian cancer (Cole, C. et al. Cancer Biol Ther 10, 2010), head and neck cancer (Koole, K. et al. Virchows Arch 469, S31-S31, 2016), and pancreatic cancer (Ishiwata, T. et al. Am J Pathol 180, 1928-1941, 2012).

### DETAILED DESCRIPTION OF THE INVENTION

### Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

A C₁-C₆ alkyl in the context of the present specification signifies a saturated linear or branched hydrocarbon having 1, 2, 3, 4, 5 or 6 carbon atoms, wherein one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples for a C₁-C₆ alkyl include the examples given for C₁-C₄ alkyl above, and additionally 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-inyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety.

The term C₃-C₇ cycloalkyl in the context of the present specification relates to a saturated hydrocarbon ring having 3, 4, 5, 6 or 7 carbon atoms, wherein in certain embodiments, one carbon-carbon bond may be unsaturated. Non-limiting examples of a C₃-C₇ cycloalkyl moiety include cyclopropanyl (—C₃H₅), cyclobutanyl (—C₄H₇), cyclopentenyl (C₅H₉), and cyclohexenyl (C₆H₁₁) moieties. In certain embodiments, a cycloalkyl is substituted by one C₁ to C₄ unsubstituted alkyl moiety. In certain embodiments, a cycloalkyl is substituted by more than one C₁ to C₄ unsubstituted alkyl moieties.

The term carbocycle in the context of the present specification relates to a cyclic moiety composed of carbon and hydrogen atoms only. An aromatic carbocycle is also named aryl. A non-aromatic carbocycle is also named cycloalkyl.

The term heterocycle in the context of the present specification relates to a cyclic moiety, wherein at least one ring atom is replaced or several ring atoms are replaced by a nitrogen, oxygen and/or sulphur atom. An aromatic heterocycle is also named heteroaryl. A non-aromatic heterocycle is a cycloalkyl, wherein at least one ring atom is replaced or several ring atoms are replaced by a nitrogen, oxygen and/or sulphur atom.

The term heterobicycle in the context of the present specification relates to two directly connected cycles, wherein at least one ring atom is replaced or several ring atoms are replaced by a nitrogen, oxygen and/or sulphur atom. A heterobicycle is composed of two heterocycles or of one heterocycle and one carbocycle.

The term unsubstituted Cₙ alkyl when used herein in the narrowest sense relates to the moiety —CₙH₂ₙ— if used as a bridge between moieties of the molecule, or —CₙH₂ₙ₊₁ if used in the context of a terminal moiety.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable—linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, chloride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term hydroxyl substituted group refers to a group that is modified by one or several hydroxyl groups OH.

The term amino substituted group refers to a group that is modified by one or several amino groups NH₂.

The term carboxyl substituted group refers to a group that is modified by one or several carboxyl groups COOH.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —(CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂ and —CH₂CH(CH₂NHEt)₂ for terminal moieties and —CH₂CHNH₂—, —CH₂CHNHMe-, —CH₂CHNHEt-for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

The term sulfoxyl substituted group refers to a group that is modified by one or several sulfoxyl groups —SO₂R, or derivatives thereof, with R having the meaning as laid out in the preceding paragraph and different from other meanings assigned to R in the body of this specification.

The term amine substituted group refers to a group that is modified by one or several amine groups —NHR or —NR₂, or derivatives thereof, with R having the meaning as laid out in the preceding paragraph and different from other meanings assigned to R in the body of this specification.

The term carbonyl substituted group refers to a group that is modified by one or several carbonyl groups —COR, or derivatives thereof, with R having the meaning as laid out in the preceding paragraph and different from other meanings assigned to R in the body of this specification.

An ester refers to a group that is modified by one or several ester groups —CO₂R, with R being defined further in the description.

An amide refers to a group that is modified by one or several amide groups —CONHR, with R being defined further in the description.

The term halogen-substituted group refers to a group that is modified by one or several halogen atoms selected (independently) from F, Cl, Br, I.

The term fluoro substituted alkyl refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

Non-limiting examples of hydroxyl- and fluoro-substituted alkyl include —CHFCH₂OH, —CF₂CH₂OH, —(CHF)₂CH₂OH, —(CF₂)₂CH₂OH, —(CHF)₃CH₂OH, —(CF₂)₃CH₂OH, —(CH₂)₃OH, —CF₂CH(OH)CH₃, —CF₂CH(OH)CF₃, —CF(CH₂OH)CHFCH₃, and —CF(CH₂OH)CHFCF₃.

The term aryl in the context of the present specification signifies a cyclic aromatic C₅-C₁₀ hydrocarbon. Examples of aryl include, without being restricted to, phenyl and naphthyl.

A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. An aryl or a heteroaryl in the context of the specification additionally may be substituted by one or more alkyl groups.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease.

The term metastasis in the context of the present specification relates to the dissemination and growth of neoplastic cells outside the original tumor bed in the same organ or in an organ distant from that in which they originated. In particular embodiments, the treatment or prevention with the disclosed compounds is employed for metastasis associated with aberrant FGFR signalling. The compounds of the invention specifically reduce the motile behaviour of metastatic cells and reduce dissemination. In particular embodiments, the compounds of the invention are employed for prevention or treatment of motility and dissemination of cancerous cells.

### FGFR-Driven Tumorigenesis

Approximately 7% of all human tumors harbor an FGFR alteration (66% gene amplification, 26% mutations, 8% gene rearrangements) (Helsten, T. et al., Clin Cancer Res., 259-268 (2016) doi:10.1158/1078-0432.CCR-14-3212). FGFR1 is frequently amplified in 20-25% of squamous non-small cell lung cancer (Weiss, J. et al., Science Translational Medicine (2010) doi:10.1126/scitranslmed.3001451) and 15% breast cancer (Andre, F. et al., Clin Cancer Res., 15, 441-452 (2009)) and mutated in 18% of midline gliomas (Di Stefano, A. L. et al., Journal of Clinical Oncology 36, 2005 (2018)). FGFR2 is mainly activated by gene fusions in intrahepatic cholangiocarcinomas (iCCA, 15%) and mutations in 10% of endometrial tumors have also been described (Konecny, G. E. et al., The Lancet Oncology 16, 686-694 (2015); Verlingue, L. et al., European Journal of Cancer 87, 122-130 (2017).). FGFR3 is affected by mutations in urothelial carcinomas (up to 20% in the metastatic setting⁷); gene fusions (mainly FGFR3-TACC3) are present in glioblastomas and gliomas (3-6% (Di Stefano, A. L. et al., Journal of Clinical Oncology 36, 2005 (2018); Singh, D. et al., Science 337, 1231-1235 (2012); Di Stefano, A. L. et al., Clinical Cancer Research 21, 3307-3317 (2015))), as well as in bladder cancer (2-3% (Robertson, A. G. et al., Cell 171, 540-556.e25 (2017))). FGFR1-4 signal via Fibroblast Growth Factor Receptor Substrate 2 (FRS2)-dependent (RAS/MAPK and PI3K/AKT) and FRS2-independent (PLC-γ, JAK-STAT) pathways (Turner, N. & Grose, Nat Rev Cancer, 1-14 (2010) doi:10.1038/nrc2780). FRS2 interacts with FGFRS via its phosphotyrosine binding domain (PTB) (Gotoh, N., Cancer Science 99, 1319-1325 (2008)) and increased expression or activation for FRS2 is involved in tumorigenesis of several tumor entities (Zhang, K. et al., Cancer Research 73, 1298-1307 (2013); Li, J.-L. & Luo, European review for medical and pharmacological sciences 24, 97-108 (2020); Wu, S. et al., Nature Communications 1-12 (2019) doi:10.1038/s41467-019-08576-5; Liu, J. et al., Oncogene, 35, 1750-1759 (2015); Chew, N. J. et al., Cell Communication and Signaling 18, 1-17 (2020)). Targeting of FRS2 function via repressing the FRS2-directed N-Myristoyltransferase repressed FGFR signaling, cell proliferation and migration in several cancer types (Li, Q. et al., The Journal of biological chemistry 293, 6434-6448 (2018)). Pharmacological inhibition of FGFRs reduces brain invasion in medulloblastoma and reduces metastasis in hepatocellular carcinoma (Huynh, H. et al., Hepatology 69, 943-958 (2019)) and lung cancer (Preusser, M. et al., Lung Cancer 83, 83-89 (2014)). FGFR-driven invasiveness depends on FRS2 (Huynh, H. et al., Hepatology 69, 943-958 (2019)). The FGF ligands of FGFRS are highly expressed in skeletal muscle (Pedersen, B. K. & Febbraio, M. A., Nature Reviews Endocrinology vol. 8 457-465 (2012)), bone (Su, N., Du, X. L. & Chen, Frontiers in Bioscience vol. 13 2842-2865 (2008)) and in CSF-secreting choroid plexus (Greenwood, S. et al., Cerebrospinal Fluid Research 5, 13-20 (2008)) and can serve as chemokinetic and chemotactic factors driving local invasion and distal spread. Repression of FGFR-FRS2 signaling may thus not only suppress the proliferative potential of tumor cells but also halt their metastatic spread driven by chemokinetic or chemotactic functions of secreted FGFs in the primary tumor and the target organ, respectively.

Selective (for example AZD4547, NVP-BGJ398 and JNJ-42756493) and non-selective (for example dovitinib or ponatinib) FGFR inhibitors have been explored for cancer therapy (Facchinetti, F. et al., Clin Cancer Res, (2020) doi:10.1158/1078-0432.CCR-19-2035; Yamaoka, T. et al., Int. J. Mol. Sci. 19, 1-35 (2018)). Resistance to FGFR inhibitors can evolve similarly as to other RTK inhibitors, either by the formation of gatekeeper mutations in the catalytic domain or the activation of alternative RTKs, which enable bypass mechanism for downstream signaling activation (Yamaoka, T., et al., Int. J. Mol. Sci. 19, 1-35 (2018)). Such mutations in FGFRS can occur in the ATP binding cleft and may create a steric conflict to limit drug-binding efficacy. Examples include FGFR3_V555M, FGFR1_V561 and FGFR2_V564, which induce resistance to FGFR inhibitors in vitro (Chell, V. et al., Oncogene 32, 3059-3070 (2013); Byron S. A. et al., Neoplasia 15, 975-988 (2013)).

The inventors' approach to target the non-enzymatically active FGFR adaptor protein FRS2 could prevent the evolution of FGFR gatekeeper mutations or help overcoming the resistance of gatekeeper FGFR-driven tumors by blocking signaling downstream of the RTK. Targeting FRS2 is likely also effective against tumors driven by the FGFR3-TACC3 fusion, where FRS2 is phosphorylated and transmits signaling to the oncogenic MAP kinase pathway (Chew, N. J. et al., Cell Communication and Signaling 18, 1-17 (2020)). Furthermore, toxicities related to FGFR inhibitor treatments have been reported and include hyper-phosphoremia, fatigue, dry skin and mouth with stomatitis, hand-foot syndrome and gastrointestinal dysfunctions (Facchinetti, F. et al., Clin Cancer Res, (2020) doi:10.1158/1078-0432.CCR-19-2035). An approach specifically targeting FRS2 with limited off-target compound activities may reduce the severity of toxicities currently associated with FGFR inhibition.

A first aspect of the invention relates to a compound of the general formula (500) for use in treatment or prevention of metastasis

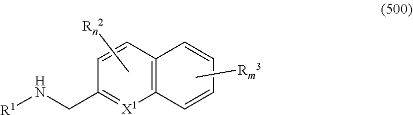

wherein
   X¹ is selected from N, O, and S, particularly X¹ is N,
   R¹ is selected from a (linear or branched) C₁-C₁₆ alkyl, (linear or branched) C₂-C₁₆ alkene, heteroaryl, aryl, a C₄-C₇ cyclo-alkyl, and a C₃-C₆ heterocycle, wherein R¹ is unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and unsubstituted or substituted C₁-C₅ alkyl or C₂-C₅ alkene, particularly R¹ is substituted with one moiety selected from OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
   each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
   n is 0, 1, 2, or 3, particularly n is 1;
   m is 0, 1, 2, 3, or 4, particularly m is 1 or 2.

In certain embodiments, R¹ is —CH₂—NH—CHR⁴R⁵, wherein
R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
or
R⁴ and R⁵ together form an unsubstituted or OH—, halogen-, and/or CN-substituted cyclo-pentane or cyclo-hexane.

In certain embodiments, the compound is of the general formula (700)

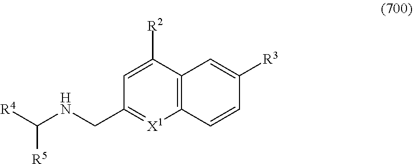

wherein
each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
R⁴ and R⁵ have the same definition as in claim 2;
X¹ is selected from N, O, and S, particularly X¹ is N.

In certain embodiments, R⁴ is selected from unsubstituted C₁-C₅ alkyl and C₂-C₅ alkene and R⁵ is an electronegative moiety selected from C₁-C₅ alkyl and C₂-C₅ alkene substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl.

In certain embodiments, R⁴ is selected from ethyl, iso-propyl, and tert-butyl.

In certain embodiments, R⁵ is selected from OH—, halogen-, and/or CN-substituted methyl, ethyl, and isopropyl.

In certain embodiments, R² is selected from C₁-C₃ alkyl, OH, NH₂, and halogen, particularly F or Cl.

In certain embodiments, R² is selected from C₁-C₃ alkyl, and OH.

In certain embodiments, R³ is selected from OH, NH₂, and halogen. In certain embodiments, R³ is halogen, more particularly R³ is F.

In certain embodiments, X¹ is N.

In certain embodiments, the metastasis arises from a cancer selected from bladder cancer, pediatric brain tumour, medulloblastoma, multiple myeloma, colorectal cancer and gastric cancer.

A second aspect of the invention relates to a compound as described in the first aspect for use as an angiogenesis antagonist. In certain embodiments, the angiogenesis antagonist is provided in treatment or prevention of cancer. In certain embodiments, the cancer is selected from bladder cancer, hepatocellular carcinoma, and prostate cancer.

A third aspect of the invention relates to a compound as described in the first aspect for use in prevention or treatment of an FGFR-driven disease.

A fourth aspect of the invention relates to a compound of the general formula (700)

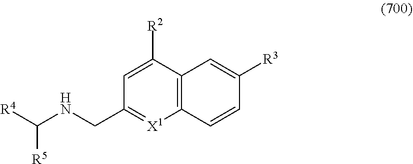

wherein
each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
   or
R⁴ and R⁵ together form an unsubstituted or OH—, halogen-, and/or CN-substituted cyclo-pentane or cyclo-hexane;
X¹ is selected from N, O, and S, particularly X¹ is N,
with the proviso that the compound is not characterized by the formula (001),

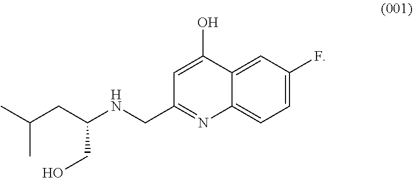 [SMILES: CC(C)C[C@@H](CO)NCc1cc(O)c2cc(F)ccc2n1]

The bicyclic structure interacts with the glycine and arginine rich regions on the target protein. The bicyclic structure interacts specifically with R152, R153, R137, G157, G159 and G151.

Particularly X¹ is a heteroatom. The heteroatom (N, O or S) establishes a first interaction with the target protein.

In certain embodiments, R⁴ is selected from unsubstituted C₁-C₅ alkyl and C₂-C₅ alkene and R⁵ is an electronegative moiety selected from C₁-C₅ alkyl and C₂-C₅ alkene substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl.

The electronegative moiety R⁵ establishes an interaction with the lysine-rich region of the target protein. R⁵ interacts particularly with L121 and L141.

In certain embodiments, R⁴ is selected from ethyl, iso-propyl, and tert-butyl, and R⁵ is selected from OH—, halogen-, and/or CN-substituted methyl, ethyl, and isopropyl.

In certain embodiments, R² is selected from C₁-C₃ alkyl, OH, NH₂, and halogen, particularly F or Cl. In certain embodiments, R² is selected from C₁-C₃ alkyl, and OH.

In certain embodiments, R³ is selected from OH, NH₂, and halogen. In certain embodiments, R³ is halogen, more particularly R³ is F.

In certain embodiments, X¹ is N.

A fifth aspect of the invention relates to a compound according to the fourth aspect for use as a medicament with the proviso that the compound includes the compound characterized by formula (001),

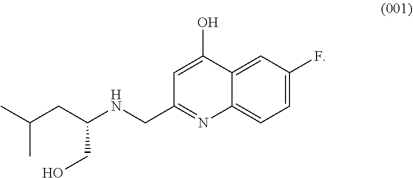 [SMILES: CC(C)C[C@@H](CO)NCc1cc(O)c2cc(F)ccc2n1]

A sixth aspect of the invention relates to a compound according to the fourth aspect for use in treatment or prevention of cancer with the proviso that the compound includes the compound characterized by the formula (001). In certain embodiments, the cancer is selected from ependymoma, prostate cancer, esophageal cancer, thyroid cancer, hepatocellular carcinoma, testicular cancer, pediatric brain tumour, medulloblastoma, rhabdomyosarcoma, gastric cancer, pulmonary pleomorphic carcinoma, breast cancer, non-small cell lung cancer, liposarcoma, cervical cancer, colorectal cancer, melanoma, multiple myeloma, endometrial cancer, bladder cancer, glioblastoma, squamous cell carcinoma of the lung, ovarian cancer, head and neck cancer, and pancreatic cancer, sarcoma. In certain embodiments, the cancer is selected from bladder cancer, multiple myeloma, gastric cancer, pediatric brain tumour, medulloblastoma, glioblastoma, ependymoma, colorectal cancer and sarcoma. In certain embodiments, the cancer is selected from bladder cancer, pediatric brain tumour, medulloblastoma, multiple myeloma, colorectal cancer and gastric cancer.

Medical treatment, Dosage Forms and Salts

Similarly, within the scope of the present invention is a method or treating cancer or metastasis in a patient in need thereof, comprising administering to the patient a compound according to the above description.

Similarly, a dosage form for the prevention or treatment of cancer is provided, comprising a non-agonist ligand or antisense molecule according to any of the above aspects or embodiments of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Topical administration is also within the scope of the advantageous uses of the invention. The skilled artisan is aware of a broad range of possible recipes for providing topical formulations, as exemplified by the content of Benson and Watkinson (Eds.), Topical and Transdermal Drug Delivery: Principles and Practice (1st Edition, Wiley 2011, ISBN-13: 978-0470450291); and Guy and Handcraft: Transdermal Drug Delivery Systems: Revised and Expanded (2^{nd} Ed., CRC Press 2002, ISBN-13: 978-0824708610); Osborne and Amann (Eds.): Topical Drug Delivery Formulations (1^{st} Ed. CRC Press 1989; ISBN-13: 978-0824781835).

### Pharmaceutical Composition and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a compound as identified herein, or its pharmaceutically acceptable salt, as specified in detail above, for use in a method of manufacture of a medicament for the treatment or prevention of cancer or metastasis.

Similarly, the invention encompasses methods of treatment of a patient having been diagnosed with a disease associated with cancer or metastasis. This method entails administering to the patient an effective amount of a compound as identified herein, or its pharmaceutically acceptable salt, as specified in detail herein.

Wherever alternatives for single separable features such as, for example, a ligand type or medical indication are laid out herein as “embodiments”, it is to be understood that such alternatives may be combined freely to form discrete embodiments of the invention disclosed herein. Thus, any of the alternative embodiments for a ligand type may be combined with any medical indication mentioned herein.

The invention further encompasses the following items.

### Items

1. A compound of the general formula (100)

R¹-L-BC (100)
wherein
   L is a linker consisting of a C₁-C₆ alkyl, a C₁-C₅ amine, or a C₁-C₄ amide, wherein L is unsubstituted or substituted with C₁-C₄ alkyl, particularly L is a C₁-C₅ amine;
   R¹ is selected from a (linear or branched) C₁-C₁₆ alkyl, (linear or branched) C₂-C₁₆ alkene, heteroaryl, aryl, a C₄-C₇ cyclo-alkyl, and a C₃-C₆ heterocycle, wherein R¹ is unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl,
   particularly R¹ is substituted with one moiety selected from OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
   BC is an aromatic or non-aromatic hetero-bicycle or carbobicycle, which is unsubstituted or substituted with 1-7 moieties independently selected from C₁-C₃ alkyl, OR^{OH}, NH₂, CN, COOR^{COO} and halogen, with R^{COO} and R^{OH} being independently selected from H, and C₁-C₃ alkyl;
   with the proviso that the compound is not characterized by the formula (001),

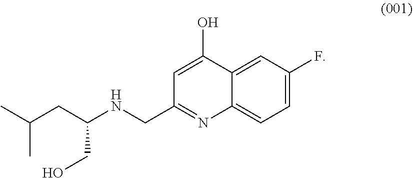 [SMILES: CC(C)C[C@@H](CO)NCc1cc(O)c2cc(F)ccc2n1]

2. The compound according to item 1 of the general formula (201), (202), or (203)

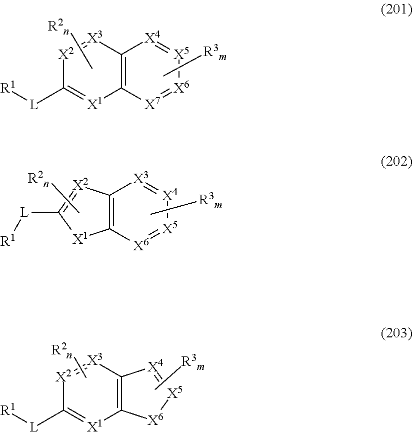

wherein
   L and R¹ have the same meanings as defined in item 1;
   n is 0, 1, 2, or 3, particularly n is 1;
   m is 0, 1, 2, 3, or 4, particularly m is 1 or 2;
   each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
   each X¹-X⁷ is independently selected from CH, N, O, and S, wherein 2-7 atoms of X¹-X⁷ are CH,
   particularly X¹ is selected from N, O, and S, and all other X are CH,
   more particularly X¹ is N, and all other X are CH.
3. The compound according to item 1 or 2 of the general formula (300)

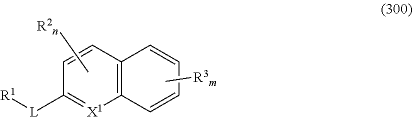

wherein
   X¹, L, R¹, R², R³, n, and m have the same meanings as defined in item 1 or 2.
4. The compound according to any one of the preceding items of the general formula (400)

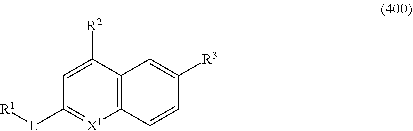

wherein
   X¹, L, R¹, R², and R³ have the same meanings as defined in item 1 or 2.
5. The compound according to any one of the preceding items 1 to 3 of the general formula (500)

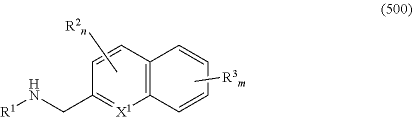

wherein
   X¹, R¹, R², R³, n, and m have the same meanings as defined in item 1 or 2.
6. The compound according to any one of the preceding items 1 to 3 of the general formula (600)

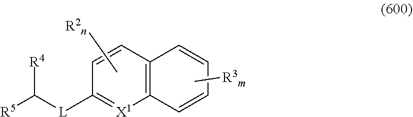

wherein
   X¹, L, R¹, R², R³, n, and m have the same meanings as defined in item 1 or 2;
   R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
   or
   R⁴ and R⁵ together form an unsubstituted or OH—, halogen-, and/or CN-substituted cyclo-pentane or cyclo-hexane.
7. The compound according to any one of the preceding items of the general formula (700)

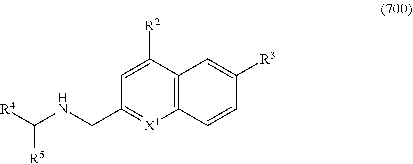

wherein
   X¹, R², R³, R⁴, and R⁵ have the same meanings as defined in item 1, 2 or 6.
8. The compound according to any one of the preceding items 1-4 or 6, wherein L has a length of 1-6 atoms, more particularly L has a length of 2-4 atoms, most particularly L has a length of 2 atoms.
9. The compound according to any one of the preceding items 6 or 7, wherein R⁴ is selected from unsubstituted C₁-C₅ alkyl and C₂-C₅ alkene and R⁵ is an electronegative moiety selected from C₁-C₅ alkyl and C₂-C₅ alkene substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl particularly R⁴ is selected from ethyl, iso-propyl, and tert-butyl, and R⁵ is selected from OH—, halogen-, and/or CN-substituted methyl, ethyl, and isopropyl;
10. The compound according to any one of the preceding items, wherein R² is selected from C₁-C₃ alkyl, OH, NH₂, and halogen, particularly F or Cl, particularly R² is selected from C₁-C₃ alkyl, and OH.
11. The compound according to any one of the preceding items, wherein R³ is selected from OH, NH₂, and halogen, particularly R³ is halogen.
12. The compound according to any one of the preceding items, wherein X¹ is N.
13. A compound according to any one of the preceding items, for use as a medicament with the proviso that the compound includes the compound characterized by formula (001),

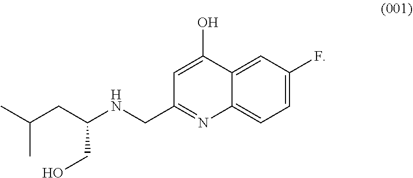 [SMILES: CC(C)C[C@@H](CO)NCc1cc(O)c2cc(F)ccc2n1]

14. The compound as described in any of the preceding items for use in treatment or prevention of cancer, particularly wherein said cancer is selected from ependymoma, prostate cancer, esophageal cancer, thyroid cancer, hepatocellular carcinoma, testicular cancer, pediatric brain tumour, medulloblastoma, rhabdomyosarcoma, gastric cancer, pulmonary pleomorphic carcinoma, breast cancer, non-small cell lung cancer, liposarcoma, cervical cancer, colorectal cancer, melanoma, multiple myeloma, endometrial cancer, bladder cancer, glioblastoma, squamous cell carcinoma of the lung, ovarian cancer, head and neck cancer, and pancreatic cancer, sarcoma, more particularly said cancer is selected from bladder cancer, multiple myeloma, gastric cancer, pediatric brain tumour, medulloblastoma, glioblastoma, ependymoma, colorectal cancer and sarcoma, most particularly said cancer is selected from bladder cancer, pediatric brain tumour, medulloblastoma, multiple myeloma, colorectal cancer and gastric cancer with the proviso that the compound includes the compound characterized by the formula (001).
15. The compound as described in any of the preceding items 1 to 12 for use in treatment or prevention of metastasis, particularly wherein said metastasis arises from a cancer selected from bladder cancer, pediatric brain tumour, medulloblastoma, multiple myeloma, colorectal cancer and gastric cancer with the proviso that the compound includes the compound characterized by the formula (001).
16. The compound as described in any of the preceding items 1 to 12 for use as an angiogenesis antagonist, particularly an angiogenesis antagonist in treatment or prevention of cancer, more particularly wherein said cancer is selected from bladder cancer, hepatocellular carcinoma, and prostate cancer, with the proviso that the compound includes the compound characterized by the formula (001).

The invention is further illustrated by the following examples and figures, from which further embodiments and advantages can be drawn. These examples are meant to illustrate the invention but not to limit its scope.

### DESCRIPTION OF THE FIGURES

FIG. 1 shows the efficacy of the compound F3.14 determining its ability to inhibit cancer cell invasion. The graph represents the efficacy of F3.14 at 3 different concentrations—1 μM, 5 μM and 10 μM.

FIG. 2 shows the efficacy of F3.14 at 10 M.

FIG. 3 shows the binding affinities and dissociation constant (Kd) of F3.14. Nano diffraction scanning fluorimetry (nanoDSF) and Microscale thermophoresis (MST) are biophysical assays used to assess the binding of the compounds to the target protein. Any temperature shift above 1.5 degree Celsius is considered as indication for significant binding.

FIG. 4 shows the effective inhibitory concentration of F3.14—EC50 (μM).

FIG. 5 shows the biochemical specificity of F3.14 determining the ability of the compounds to inhibit FGF signalling pathway without affecting other signalling pathways. Lane 1: Control—DAOY LA-EGFP cells unstimulated, serum starved overnight and then lysed. Lane 2: bFGF (100 ng/ml)—Overnight serum starved DAOY LA-EGFP cells stimulated with bFGF for 10 minutes and then lysed. Lane 3: F3.14 (10 μM)—Overnight serum starved DAOY LA-EGFP cells treated with F3.14 for four hours, cells stimulated with bFGF for 10 minutes and then lysed.

FIG. 6 A) Binding site 1 is not involved in FGFR binding and located below the interaction site of FGFR's N-terminus with FRS2. B) Binding site 2 is the extended surface area interacting with FGFR's C-terminal end.

FIG. 7 Spheroid invasion assay using DAOY cells stimulated with bFGF+/−BGJ398 or F3-14 to determine the EC5o of F3.14.

FIG. 8 Cell titer glo assay performed with DAOY cells treated with BGJ398 or F3.14.

FIG. 9 Cell titer glo assay performed with AGS cells treated with BGJ398 or F3.14.

FIG. 10 Cell titer glo assay performed with M059K cells treated with BGJ398 or F3.14.

FIG. 11 Cell titer glo assay performed with RT112 cells treated with BGJ398 or F3.14.

FIG. 12 Cell titer glo assay performed with DMS114 cells treated with BGJ398 or F3.14.

FIG. 13 Cell titer glo assay performed with HCT116 cells treated with BGJ398 or F3.14.

FIG. 14 Cell titer glo assay performed with SKOV3 cells treated with BGJ398 or F3.14.

FIG. 15 Cell titer glo assay performed with SNU16 cells treated with BGJ398 or F3.14.

FIG. 16 Table showing the in vitro absorption, distribution, metabolism, elimination and toxicity (ADMET) properties of F3.14. Efflux ration represents the permeability of F3.14, Semi-thermodynamic solubility shows the solubility of F3.14 in aqueous solutions. Intrinsic clearance and t½ shows the metabolic stability of F3.14, MTT shows the toxicity of F3.14 and potency shows the efficacy of F3.14.

FIG. 17 In vivo pharmacokinetics, 3 mice/treatment, serum concentration of compounds in μM. Table showing the in vivo pharmacokinetic (PK) properties of F3.14.

FIG. 18 Immunoblots using various FGFR-driven cell lines treated with BGJ398 or F3.14 showing the effect of the treatment on the downstream effectors of FGF signalling.

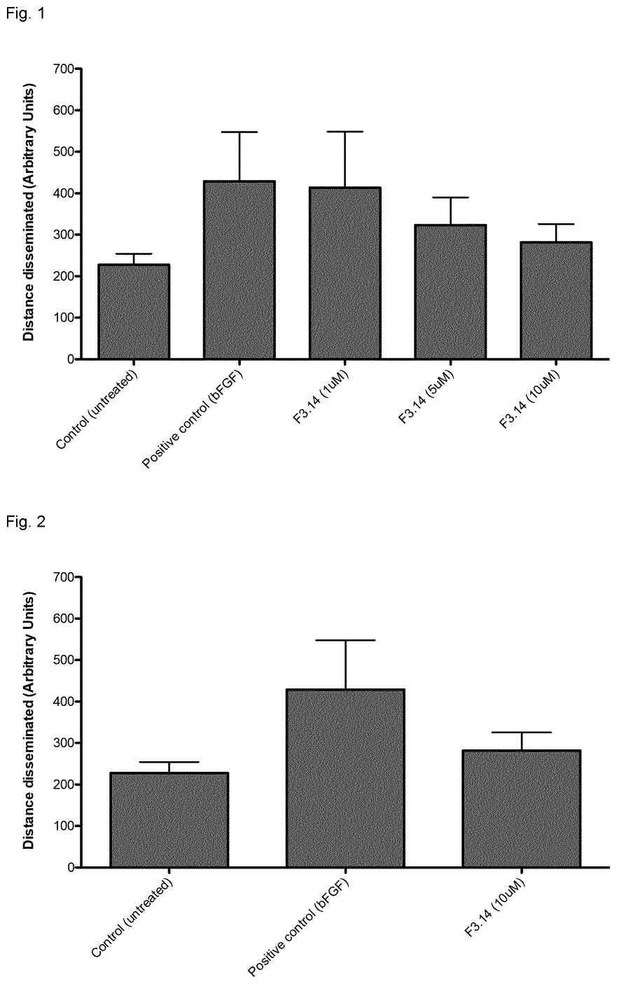

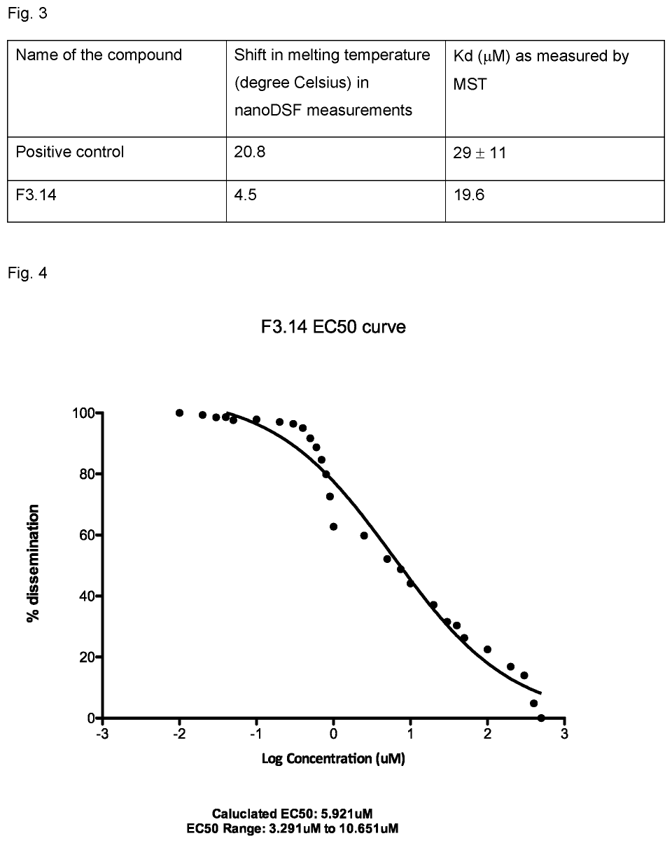

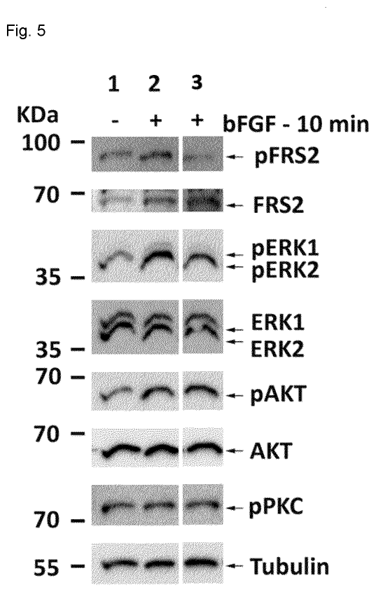

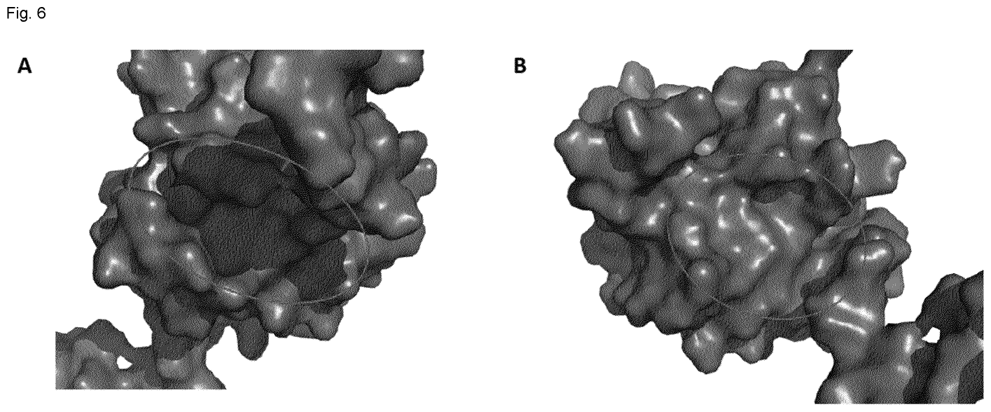

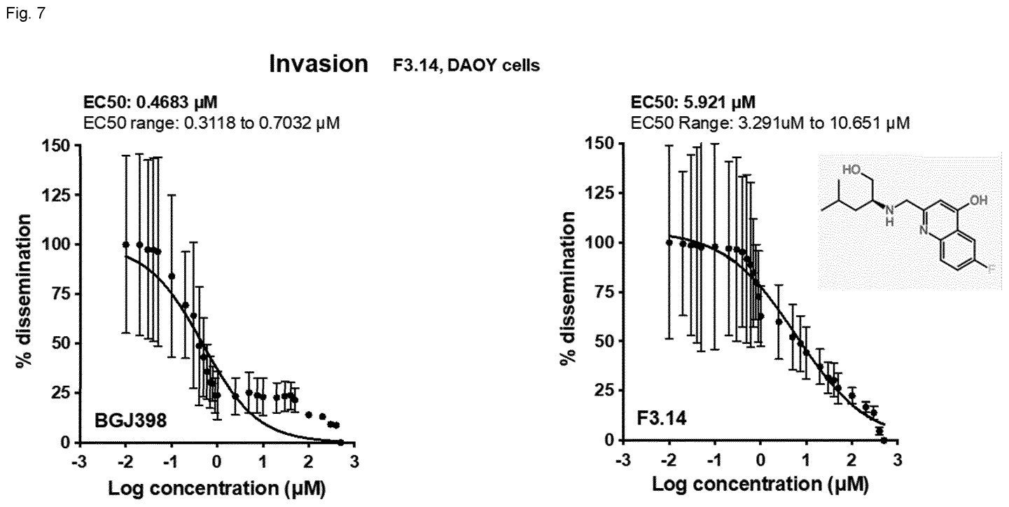

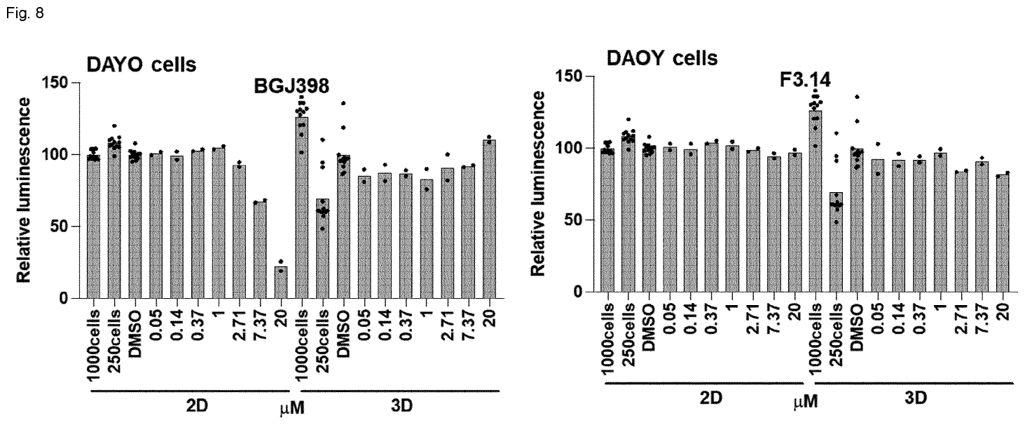

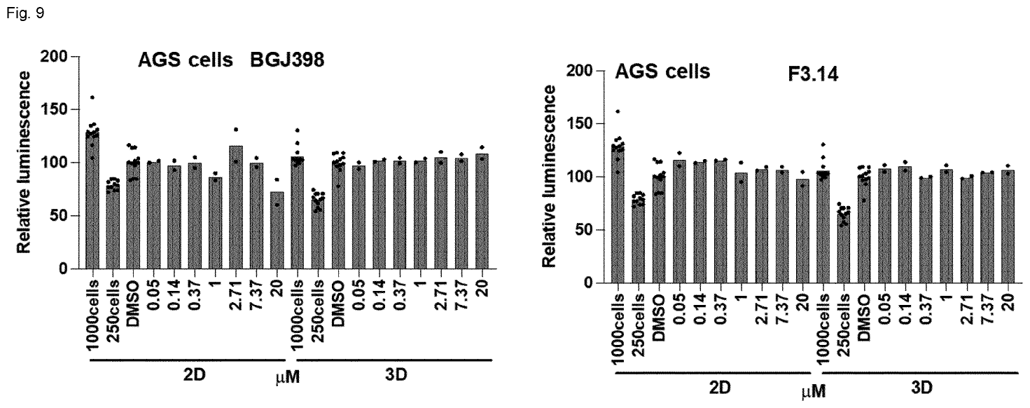

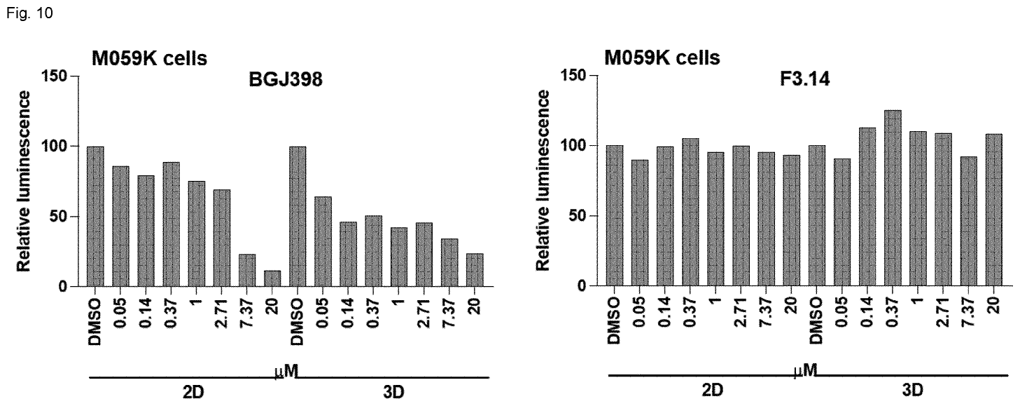

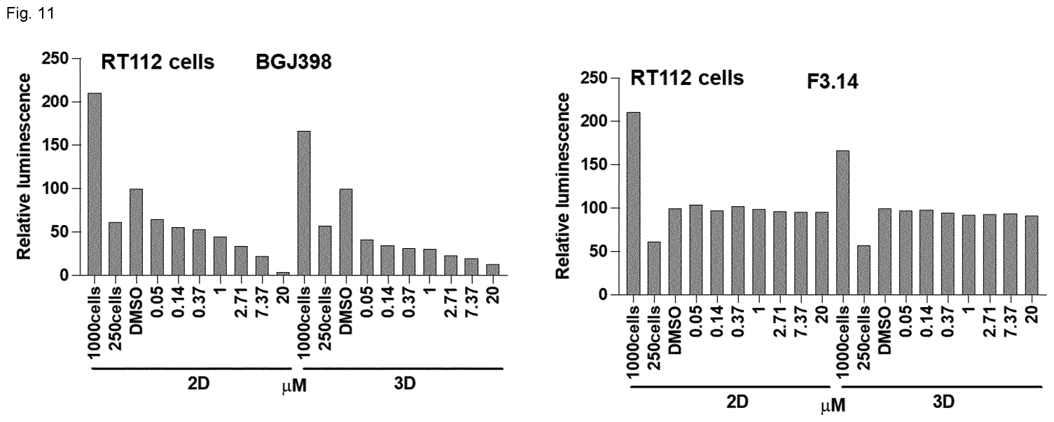

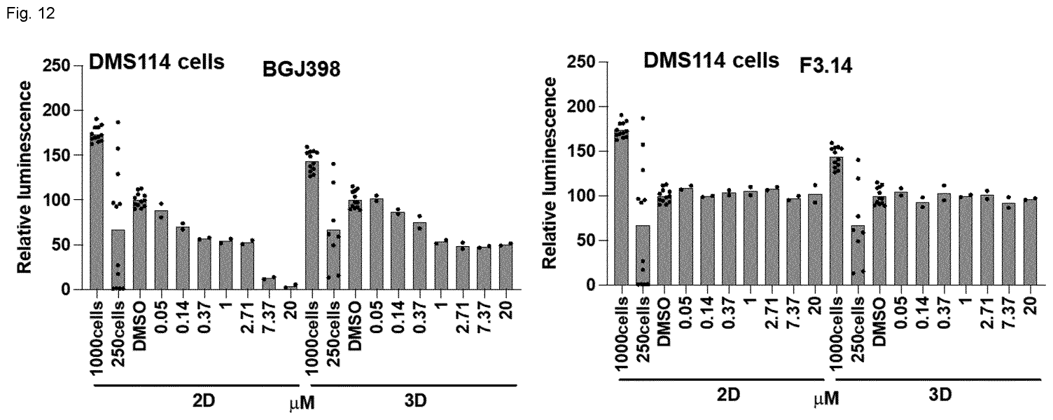

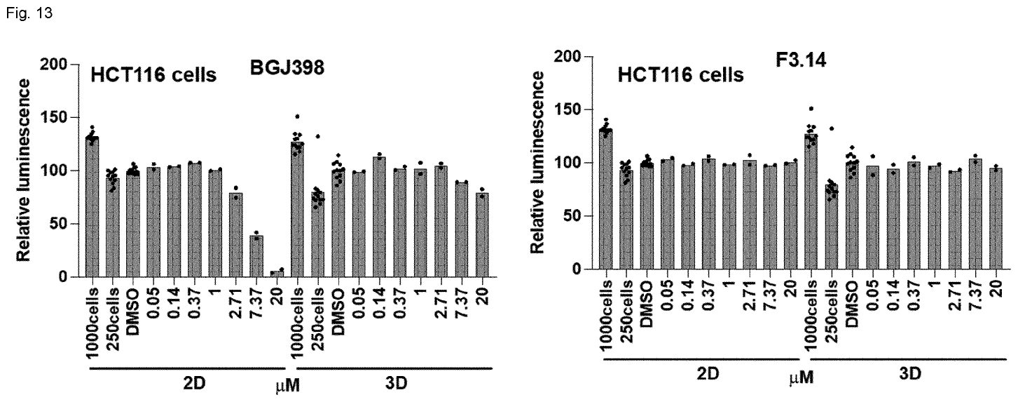

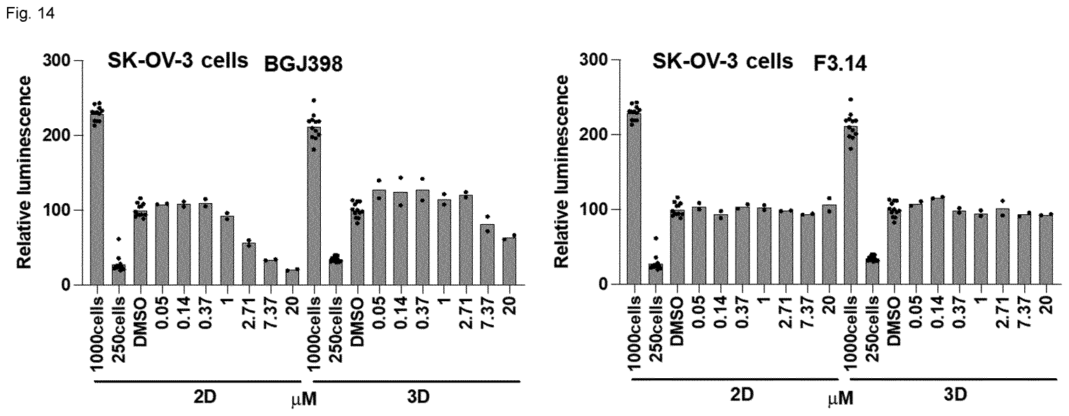

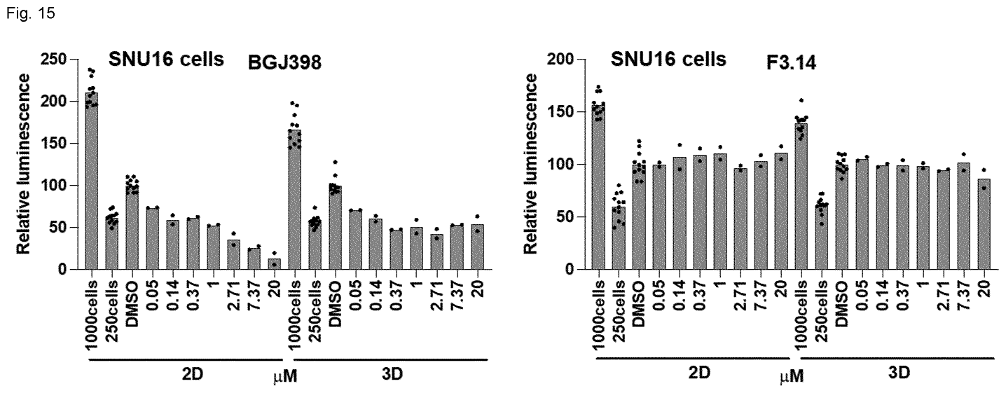

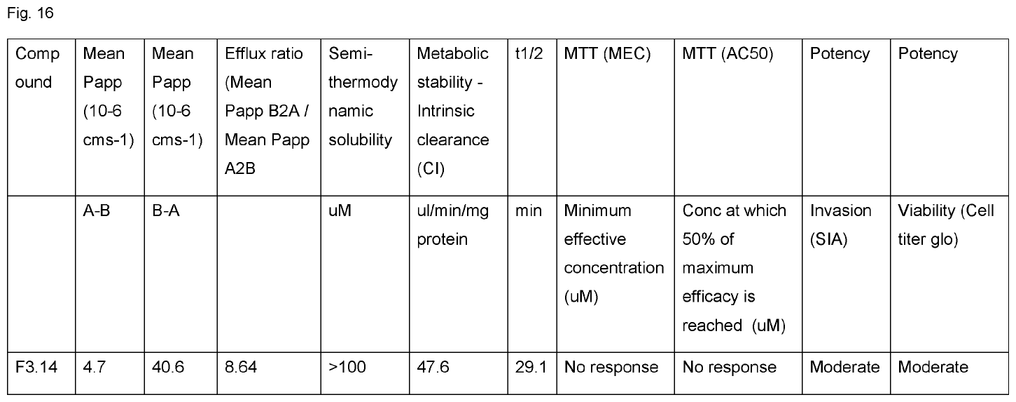

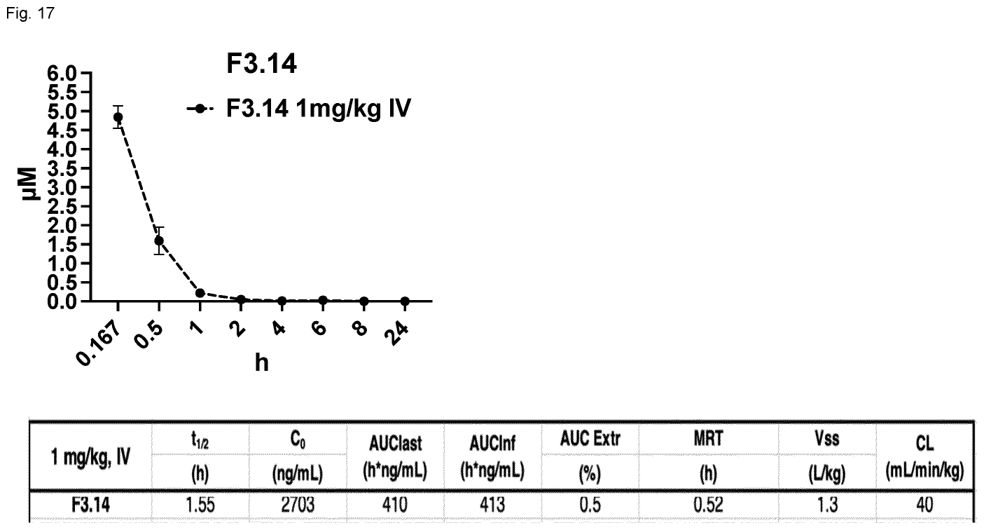

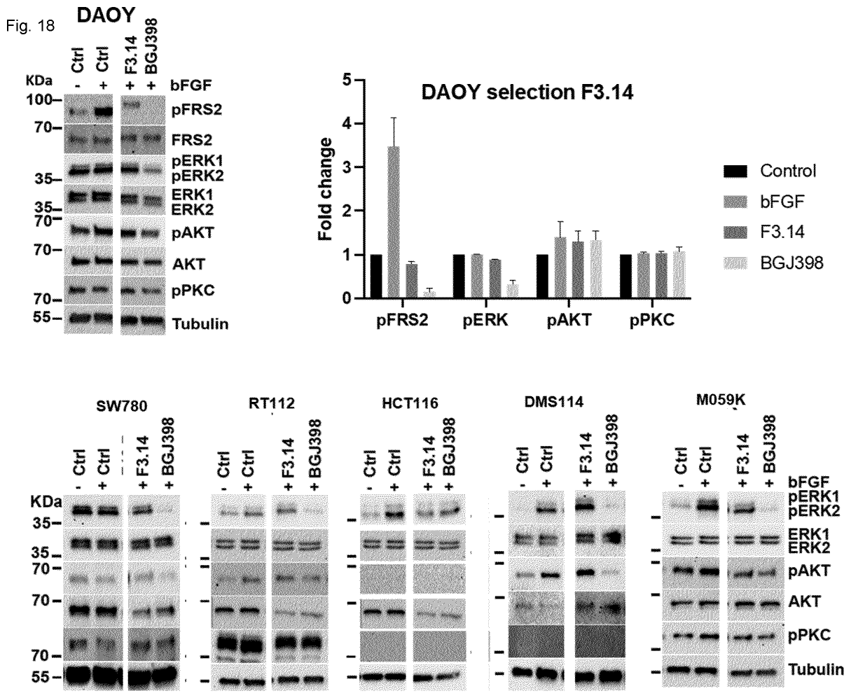

### EXAMPLES

The inventors designed an inhibitor of FRS2-FGFR interaction by screening a large library of fragments of small molecules. The inventors identified F3.14 as a putative small molecule inhibitor of FRS2-FGFR interaction. The inventors confirmed the binding of F3.14 to FRS2 using biophysical assays—nanoDSF, MST and NMR analysis. The inventors evaluated the efficacy of F3.14 in inhibiting cancer cell invasion and proliferation using FGFR-driven cancer cell models. Results from the spheroid invasion assay and cell titer glo assay show that F3.14 effectively inhibits cancer cell invasion and proliferation in all the FGFR-driven cancer cell lines tested. To test the effect of F3.14 on FGF signaling pathway, the inventors used immunoblotting. F3.14 inhibits the FGF signal transduction by inhibiting the phosphorylation of the downstream effectors of FGF signaling pathway. The inventors used in vitro ADMET studies and in vivo PK studies to determine the ‘drug-like’ properties of F3.14. Results from these assays demonstrate that F3.14 has good permeability, very good solubility, moderate intrinsic clearance, very low toxicities, and high potency. The in vivo PK studies show that F3.14 is well-tolerated in mice and could be safely administered via intravenous route to living organisms for the treatment of FGFR-driven diseases.

### Methods and Instruments

Spheroid Invasion Assay (S/A) and Automated Cell Dissemination Counter (aCDc)

1000 cells/100 μl per well were seeded in cell-repellent 96 well microplate (650790, Greiner Bio-one). The cells were incubated at 37° C. overnight to form spheroids. 70 μl of the medium were removed from each well, and remaining medium with spheroid overlaid with 2.5% bovine collagen 1. Following the polymerization of collagen, fresh medium was added to the cells and treated with growth factors and/or with inhibitors. The cells were allowed to invade the collagen matrix for 24 h, after which they were fixed with 4% PFA and stained with Hoechst. Images were acquired on an Axio Observer 2 mot plus fluorescence microscope (Zeiss, Munic, Germany) using a 5×objective. Cell invasion is determined as the average of the distance invaded by the cells from the centre of the spheroid as determined using automated cell dissemination counter (aCDc) with our cell dissemination counter software aSDlcs (Kumar et al., Sci Rep 5, 15338 (2015)).

Nano Differential Scanning Fluorimetry (nanoDSF)

Purified FRS2 protein tagged with 6× Histidine residues and Guanine nucleotide-binding protein subunit beta (GB1) was diluted in the protein buffer (100 mM sodium phosphate, 50 mM NaCl, 0.5 mM EDTA, 50 mM arginine, 1 mM TCEP, pH 7.0) to final concentration of 30 μM. The compounds were dissolved in 100% at 50 or 100 mM and further diluted to 1 mM with a final concentration of 100% DMSO. Compound and protein were mixed at 1:1 ration yielding final concentrations of 15 μM and 500 μM for the compounds. The mixture was incubated at room temperature for 15 minutes before measurement. The measurement was performed on a Prometheus system in high sensitivity capillaries. Samples were subjected to a temperature gradient of 20 to 95° C. with 1° C./min intervals.

### Microscale Thermophoresis (MST)

Purified FRS2 protein tagged with 6× Histidine residues and Guanine nucleotide-binding protein subunit beta (GB1) was labelled with 2^{nd} generation BLUE-NHS dye. The protein was labelled at a final concentration of 20 M with 60 μM dye. The labelling was performed in the protein buffer without arginine supplementation. Arginine was re-buffered to protein's buffer post-labelling. The compounds were dissolved in 100% at 50 or 100 mM and further diluted to 1 mM with a final concentration of 100% DMSO. The compounds were then diluted. In a 1:1 serial dilution from 1 mM to 61.04 nM in protein buffer supplemented with 10% DMSO. 10 μl of 50 nM labelled protein was added to 10 μl of each compound dilution for a final labelled protein concentration of 25 nM and DMSO-concentration of 5%. The samples were incubated at room temperature for 15 minutes. The experiments were performed in premium-coated capillaries. Excitation power was set at 20%, MST power to 40% (4 Kelvin temperature gradient) with a laser-on time of 20 seconds and a laser-off time of 3 seconds. Temperature was set to 25° C. Each measurement was repeated twice. The interaction was measured in two independent duplicates.

### Immunoblotting (IB)

Cancer cells were treated with bFGF (100 ng/ml) and/or with compounds and lysed using Radioimmunoprecipitation assay (RIPA) buffer. RIPA buffer lysates were resolved by SDS-PAGE and transferred to a nitrocellulose membrane using a transfer apparatus according to the manufacturer's instructions (Bio-Rad). Membranes were probed with primary antibodies against phospho-FRS2, FRS2, ERK1/2, phospho-ERK1/2, AKT, phosphor-AKT, phospho-PKC and tubulin. HRP-linked secondary antibodies (1:5000) were used to detect the primary antibodies. Chemiluminescence detection was performed using ChemiDoc Touch Gel and Western Blot imaging system (BioRad).

### Cell Titer Glo Assay

The metabolic activity and the proliferation of the cells were determined using the Cell Titer glo assay from Promega according to the manufacturer's instructions. In brief, 250 cells/100 μl/per well (for up to 72 h incubation) were seeded in Greiner Bio-One p-clear 384 well plates (655090, Greiner Bio-One) and incubated overnight at 37° C. The old media was then replaced with fresh serum-free media and the cells were treated with BGJ398 or F3.14 till the desired time point. Following appropriate incubation for each timepoint, 10 μl of the Cell titer glo reagent was added to each well (final concentration of cell titer glo reagent per well is 1:10) and incubated at 37° C. for 30 minutes. The luminescence was then measured with a signal integration time of 0.5 to 1 second per well.

### In Vivo Pharmacokinetics

3 Healthy non-SCID mice were intravenously treated with F3.14—Blood samples were collected at 2, 4, 6, 8 and 24 hours after treatment. Serum from the collected blood samples were isolated and the concentration of F3.14 in the serum was measure to determine the intrinsic clearance of F3.14.

### Pathway Analysis

RIPA buffer FGFR-driven cell lysates were resolved by SDS-PAGE and transferred to a nitrocellulose membrane using a transfer apparatus according to the manufacturer's instructions (Bio-Rad). Membranes were probed with primary antibodies against phospho-FRS2, FRS2, ERK1/2, phospho-ERK1/2, AKT, phospho-AKT, phospho-PKC and tubulin. HRP-linked secondary antibodies (1:5000) were used to detect the primary antibodies. Chemiluminescence detection was performed using ChemiDoc Touch Gel and Western Blot imaging system (BioRad). Integrated density of Immuno-reactive bands was quantified using Adobe Photoshop CS5.

### Availability of Compounds

The compound was purchased at ChemBridge under the following vendor ID: F3.14 24662310 (ChemBridge)

1. A compound of the general formula (500) for use in treatment or prevention of metastasis 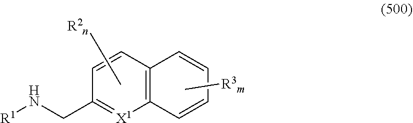
wherein
X¹ is selected from N, O, and S, particularly X¹ is N,
R¹ is selected from a (linear or branched) C₁-C₁₆ alkyl, (linear or branched) C₂-C₁₆ alkene, heteroaryl, aryl, a C₄-C₇ cyclo-alkyl, and a C₃-C₆ heterocycle, wherein R¹ is unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and unsubstituted or substituted C₁-C₅ alkyl or C₂-C₅ alkene,
particularly R¹ is substituted with one moiety selected from OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
n is 0, 1, 2, or 3, particularly n is 1;
m is 0, 1, 2, 3, or 4, particularly m is 1 or 2.

2. The compound for use according to claim 1, wherein R¹ is —CH₂—NH—CHR⁴R⁵, wherein
R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
or
R⁴ and R⁵ together form an unsubstituted or OH—, halogen-, and/or CN-substituted cyclo-pentane or cyclo-hexane.

3. The compound for use according to claim 2 of the general formula (700) 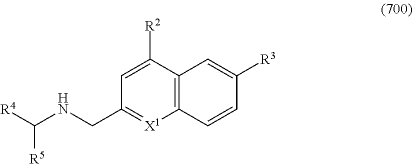
wherein
each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
X¹ is selected from N, O, and S, particularly X¹ is N.

4. The compound for use according to claim 2, wherein R⁴ is selected from unsubstituted C₁-C₅ alkyl and C₂-C₅ alkene and R⁵ is an electronegative moiety selected from C₁-C₅ alkyl and C₂-C₅ alkene substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
particularly R⁴ is selected from ethyl, iso-propyl, and tert-butyl.

5. The compound for use according to claim 2, wherein R⁵ is selected from OH—, halogen-, and/or CN-substituted methyl, ethyl, and isopropyl.

6. The compound for use according to claim 1, wherein R² is selected from C₁-C₃ alkyl, OH, NH₂, and halogen, particularly F or Cl,
particularly R² is selected from C₁-C₃ alkyl, and OH.

7. The compound for use according to claim 1, wherein R³ is selected from OH, NH₂, and halogen, particularly R³ is halogen, more particularly R³ is F.

8. The compound for use according to claim 1, wherein X¹ is N.

9. The compound for use according to claim 1, wherein said metastasis arises from a cancer selected from bladder cancer, pediatric brain tumour, medulloblastoma, multiple myeloma, colorectal cancer and gastric cancer.

10. The compound as described in claim 1 for use as an angiogenesis antagonist, particularly an angiogenesis antagonist in treatment or prevention of cancer, more particularly wherein said cancer is selected from bladder cancer, hepatocellular carcinoma, and prostate cancer.

11. The compound as described in claim 1 for use in prevention or treatment of an FGFR-driven disease.

12. A compound of the general formula (700) 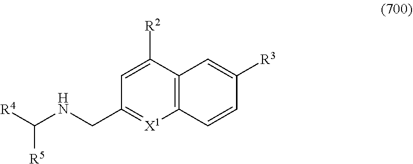
wherein
each R² and R³ is independently selected from C₁-C₃ alkyl, OR^{OH}, NH₂, CN, COOR^{COO} and halogen, with R^{COO} and R^{OH} being independently selected from H, and C₁-C₃ alkyl;
R⁴ and R⁵ are independently selected from a C₁-C₅ alkyl, C₂-C₅ alkene, wherein R⁴ and R⁵ are unsubstituted or substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
or
R⁴ and R⁵ together form an unsubstituted or OH—, halogen-, and/or CN-substituted cyclo-pentane or cyclo-hexane;
X¹ is selected from N, O, and S, particularly X¹ is N,
with the proviso that the compound is not characterized by the formula (001),

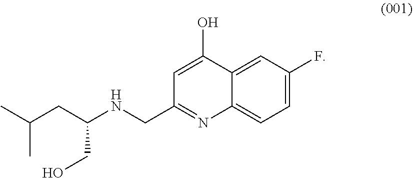 [SMILES: CC(C)C[C@@H](CO)NCc1cc(O)c2cc(F)ccc2n1]

13. The compound according to claim 12, wherein R⁴ is selected from unsubstituted C₁-C₅ alkyl and C₂-C₅ alkene and R⁵ is an electronegative moiety selected from C₁-C₅ alkyl and C₂-C₅ alkene substituted with OR^{O}, CN, halogen, NR^{N1}R^{N2}, SO₂R^{S}, COOR^{A} with R^{N1}, R^{N2}, R^{A}, R^{O}, and R^{S} being independently selected from H, and C₁-C₃ alkyl;
particularly R⁴ is selected from ethyl, iso-propyl, and tert-butyl, and R⁵ is selected from OH—, halogen-, and/or CN-substituted methyl, ethyl, and isopropyl.

14. The compound according to claim 12, wherein R² is selected from C₁-C₃ alkyl, OH, NH₂, and halogen, particularly F or Cl,
particularly R² is selected from C₁-C₃ alkyl, and OH.

15. The compound according to claim 12, wherein R³ is selected from OH, NH₂, and halogen, particularly R³ is halogen, more particularly R³ is F.

16. The compound according to claim 12, wherein X¹ is N.

17. A compound according to claim 12, for use as a medicament with the proviso that the compound includes the compound characterized by formula (001),

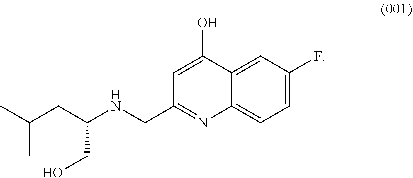 [SMILES: CC(C)C[C@@H](CO)NCc1cc(O)c2cc(F)ccc2n1]

18. A compound as described in claim 12 for use in treatment or prevention of cancer, particularly wherein said cancer is selected from ependymoma, prostate cancer, esophageal cancer, thyroid cancer, hepatocellular carcinoma, testicular cancer, pediatric brain tumour, medulloblastoma, rhabdomyosarcoma, gastric cancer, pulmonary pleomorphic carcinoma, breast cancer, non-small cell lung cancer, liposarcoma, cervical cancer, colorectal cancer, melanoma, multiple myeloma, endometrial cancer, bladder cancer, glioblastoma, squamous cell carcinoma of the lung, ovarian cancer, head and neck cancer, and pancreatic cancer, sarcoma, more particularly said cancer is selected from bladder cancer, multiple myeloma, gastric cancer, pediatric brain tumour, medulloblastoma, glioblastoma, ependymoma, colorectal cancer and sarcoma, most particularly said cancer is selected from bladder cancer, pediatric brain tumour, medulloblastoma, multiple myeloma, colorectal cancer and gastric cancer with the proviso that the compound includes the compound characterized by the formula (001).
